(19) United States
(12) Plant Patent Application Publication
(10) Pub. No.: US 2003/0014792 P1
(43) Pub. Date: Jan. 16, 2003

(54) Poplar named 'Shinsei'

(76) Inventors: Allan G. Wilkinson, Palmerston North (NZ); Lindsay E. Fung, Palmerston North (NL)

Correspondence Address: GREENLEE WINNER AND SULLIVAN P C, 5370 MANHATTAN CIRCLE, SUITE 201, BOULDER, CO, 80303, US

(21) Appl. No.: 10/159,949

(22) Filed: May 30, 2002

(30) Foreign Application Priority Data
Jun. 25, 2001 (NZ) POP006

(51) Int. Cl. A01H 5/00

(52) U.S. Cl. PLT/218

(57) ABSTRACT

A new and distinct poplar clonal cultivar, Populus nigra L.×P. maximowiczii A. Henry, is described. The cultivar results from a controlled pollination using a male P. maximowiczii from Hokkaido, Japan and a female P. nigra of unknown parentage. Both named parents are unpatented cultivars. The new cultivar is characterised by a narrow crown form, dark green foliage, high resistance to leaf rusts and anthracnose, and low palatability to possums.

### BACKGROUND OF THE INVENTION

The genus Populus consists of 30-40 named species many of which are able to hybridise with other species (within this genus). Poplars occur naturally throughout most parts of the temperate and cold regions of the Northern Hemisphere and have a long history of use by many societies in these areas.

A poplar breeding programme has been operating in New Zealand since 1968 with the aim of producing poplar varieties that are climatically suited to, and appropriate for end uses in New Zealand. This variety (or clonal cultivar) originated from this breeding programme from a controlled pollinated cross made in 1985.

This clonal cultivar has been vegetatively propagated (as unrooted 25 cm cuttings) from the original seedling identified by the code NZ 85-069-002. It has subsequently been vegetatively propagated as unrooted cuttings (25 cm), stakes (1m) and poles (2m - 3m).

It has been determined from observations of plantings of the above material that the characteristics described herein hold true to form through successive propagations.

### SUMMARY OF THE INVENTION

The clonal cultivar was created as a seedling in 1985 from a controlled pollinated cross between a female P. nigra (identified as P. nigra ‘LP1’) and pollen from a male P. maximowiczii (itself being an open pollinated seedling, from a known female parent, from Hokkaido Japan and identified as P. maximowiczii ‘73-010-072’.

The cross was carried out at The National Plant Materials Centre, Staces Road, Aokautere, Palmerston North, New Zealand,. The resulting seedlings were planted out in nursery beds at the Centre. The first vegetative propagation was from hardwood cuttings of dormant wood, this was also carried out at the National Plant Materials Centre in 1985. Following selections for further trial, cuttings were bulked up and a gene pool planting was established in 1990. Field trial plantings commenced in 1998.

The tree is characterised by good apical dominance resulting in a straight stem, with a steep branch angle giving a narrow crown form. The clonal cultivar shows high resistance to the rusts Melampsora larici-populina and M medusae and also to leaf anthracnose Marssonina brunnea. It also exhibits low palatability to possums (Trichosurus vulpecula).

In a New Zealand context this gives a distinctive variety compared with previous releases by combining narrow crown form with high pest and disease resistance. This clonal cultivar has been named Shinsei.

### BRIEF DESCRIPTION OF THE DRAWINGS

FIG. 1 shows (from left to right) upper surfaces of leaves from: 1) A fully illuminated long branch from a one-year-old cutting and, 2) a fully illuminated short branch from an eleven-year-old tree. FIG. 2 shows a fully illuminated short branch from an eleven-year-old tree. FIG. 3 shows an eleven year-old-tree planted on farmland on a sandy loam and annual rainfall of ca. 1200 mm.

FIG. 4 shows bark from the lower trunk of an eleven-year-old tree.

FIG. 5 shows leaves of the clonal cultivar (Shinsei) and its closest varieties (Eridano, Androscoggin, Oxford) from fully illuminated branches of one-year-old cuttings. Top row (from left to right): Upper (adaxial) leaf surface of Shinsei, lower (abaxial) leaf surface of Shinsei. Note moderately cordate base. Bottom row (from left to right): Upper leaf surfaces of Eridano, Androscoggin, Oxford.

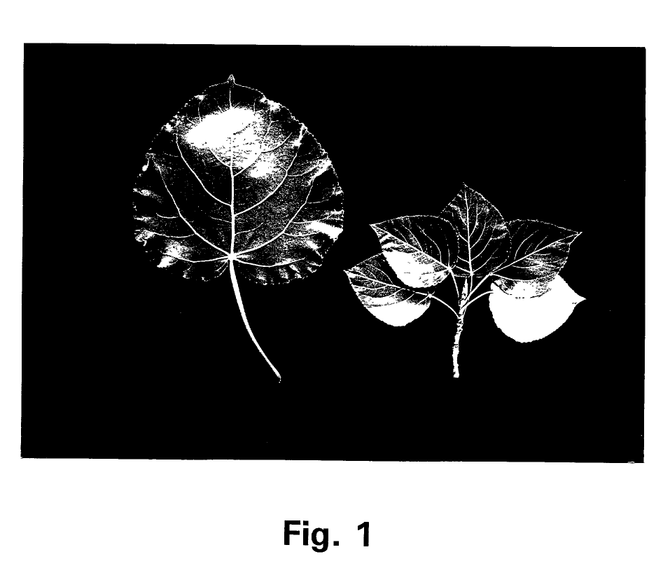

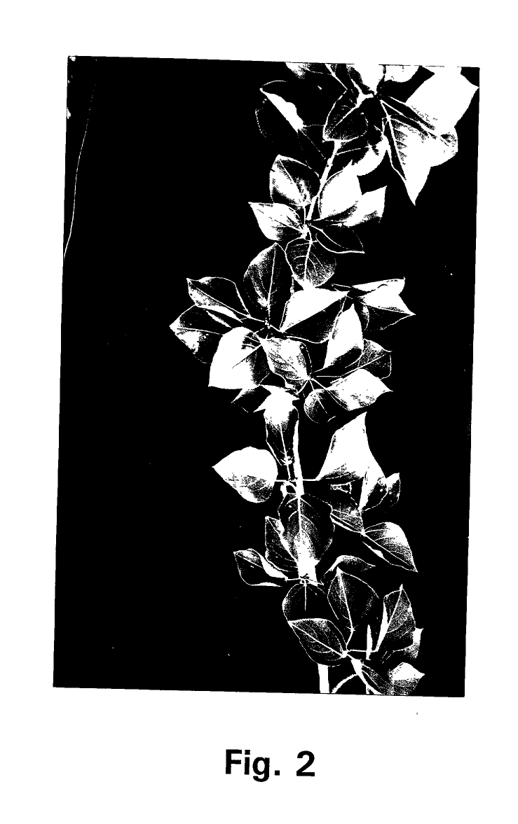

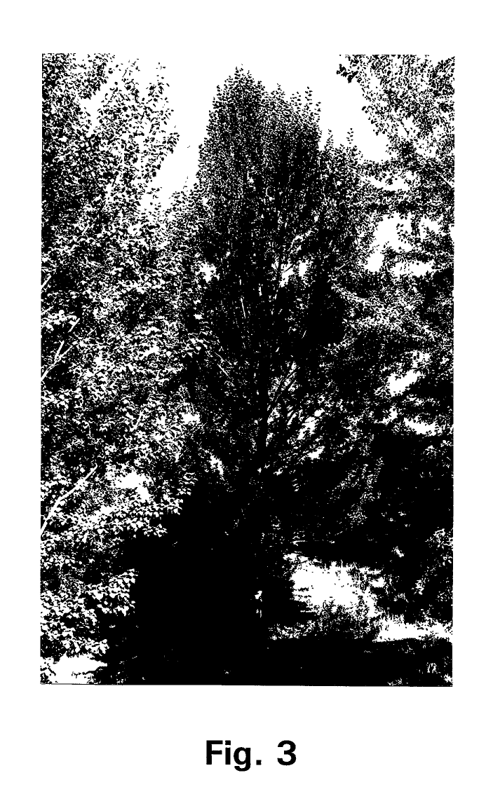

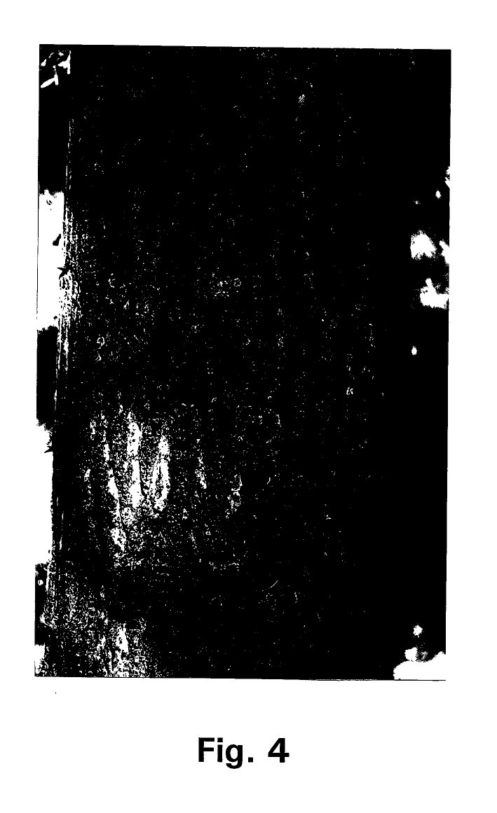

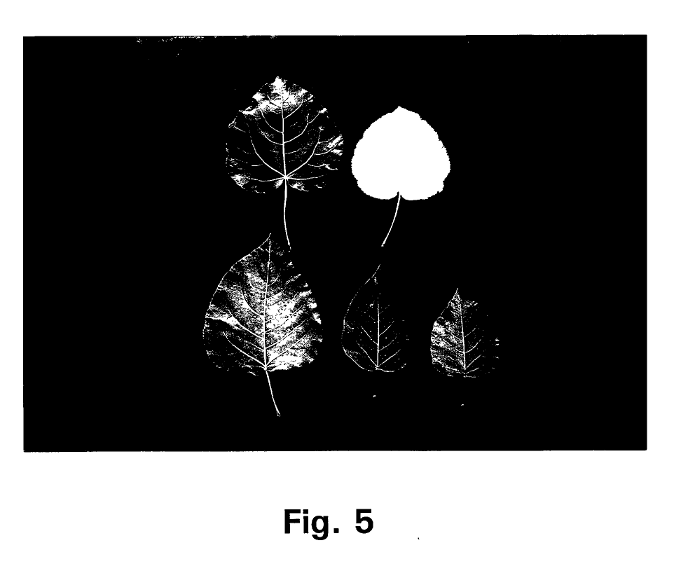

### BOTANICAL DESCRIPTION OF THE VARIETY

Young tree

As a young (1-3 years-old) tree grown from cuttings in a nursery, the clonal cultivar has a smooth, straight stem form that is circular in cross section at the base and becomes slightly angular at the tip. Stem colour is predominantly grey-green at the base, grading to a light green at the tip and lenticells (ranging in shape from round to long linear) are very prominent. Branch angle is acute with the branches tending to curve upwards.

Terminal leaf bud length is typically 20-25 mm and buds are narrow ovate in shape with a narrow acute tip. Colour is predominantly green with a red-brown tinge around the bud scale edges.

Leaf colour is typically dark-green at maturity and shape at the base is medium cordate and narrow, short acuminate at the tip, the ratio of the length of the midrib to the width of the leaf is small. Length of petiole relative to that of the midrib varies between half to similar lengths, and the petiole is light green in colour. The leaf margin is moderately undulating with small serrations. Hairs are absent from both sides of the leaf and the angle between leaf veins and the midrib are acute.

Adult tree

The oldest planted tree is almost twelve years old at the time of application and is female. Crown width in an open grown situation remains straight and non-spreading and the trunk is similarly straight. Bark on the lower trunk is furrowed at this age and light brown in colour.

Mature adult leaf form differs from the juvenile form in that the shape becomes more ovate—rhomboid due the leaf base becoming slightly cuneiform or rounded cuneiform and the leaf tip more narrow acuminate. Leaf colouration and margin characteristics remain similar to that of the young tree form, as does petiole length relative to midrib length. Vein angle becomes slightly more acute.

Growth Rates

Under nursery conditions height growth from an unrooted 25 cm cutting can be expected to reach 2-3 m in the first growing season and after two years height will be at least 6 m.

Some indicative field trial growth data are as follows:

| Age | Height (m) | Diameter at 1.4 m (cm) | Planting material |
|---|---|---|---|
| 4 | 7.1-8.2 | 7.1-9.5 | 70 cm unrooted stakes |
| 10 | 16.4 | 24.8 | 3 m unrooted pole |

Comparison to closest variety

A number of hybrids involving male P. maximowiczii have been planted in New Zealand, and these would be the closest varieties to this clone:

P. deltoides×P. maximowiczii—‘Eridano’

P. maximowiczii×P. trichocarpa‘—Androscoggin’

P. maximowiczii×P.×berolinensis—‘Oxford’

Major distinguishing features between this clone and those mentioned above are crown width in adult trees and shape of leaf base from long shoots of one-year-old cuttings and can be summarised as follows:

| | Expression of | Denomination of | |
|---|---|---|---|
| | characteristic in | the closest | Expression of characteristic in |
| Characteristic | Shinsei | variety | the named closest variety |
| Crown width | Straight (narrow) | Eridano | Spreading |
| | | Androscoggin | Spreading |
| | | Oxford | Spreading |
| Shape of leaf base* | Medium cordate | Eridano | Straight |
| | | Androscoggin | Weakly cordate |
| | | Oxford | Straight |

*from long shoots of one-year-old cuttings

We claim:

1. A new and distinct variety (clonal cultivar) of poplar tree, substantially as herein described and illustrated, characterised by a straight, narrow crown form, rapid growth and resistance to Melampsora larici-populina, M medusae (poplar leaf rusts), Marssonina brunnea (poplar leaf anthracnose), and low palatability to Trichosurus vulpecula (possum).
